(19) United States
(12) Patent Application Publication
(10) Pub. No.: US 2010/0222645 A1
(43) Pub. Date: Sep. 2, 2010

(54) HEALTH AND WELLNESS MONITORING SYSTEM

(75) Inventors: Matt NADLER, Chester, NJ (US); Alana FUTRELL, Upper Marlboro, MD (US)

Correspondence Address: VERIZON;PATENT MANAGEMENT GROUP, 1320 North Court House Road, 9th Floor, ARLINGTON, VA, 22201-2909, US

(73) Assignee: VERIZON PATENT AND LICENSING INC., Basking Ridge, NJ (US)

(21) Appl. No.: 12/394,391

(22) Filed: Feb. 27, 2009

(51) Int. Cl. A61B 5/00

(52) U.S. Cl. 600/300

(57) ABSTRACT

A method and system for monitoring wellness information method may receive wellness information from a mobile device. The received wellness information may be compared against a monitoring profile associated with the mobile device, wherein the monitoring profile includes a number of rules, each rule including defined criteria and a result event to be executed when the defined criteria are met. It may be determined whether the received wellness information matches the defined criteria in one of the number of rules. The result event may be executed when the received wellness information matches the defined criteria in one of the number of rules.

### BACKGROUND

The ability to adequately care for loved ones in an ever more busy world is a prevailing concern in today's society, particularly when considering the large number of “baby boomer” generation members rapidly approaching old age. Further, unlike historical family units, it is increasingly common for loved ones, who are caregivers or support providers, to live in geographically disparate areas, sometimes even in different countries.

Mobile consumer electronic devices, such as mobile phones, personal digital assistants (PDAs), portable gaming devices, and other electronic devices have become increasingly ubiquitous, and the functionality available to such devices continues to increase. The ever-present nature and ease of use of such devices has enabled geographically separated loved ones, caregivers or supper providers to communicate simply and easily.

### BRIEF DESCRIPTION OF THE DRAWINGS

FIG. 1 illustrates an exemplary system in which systems and methods described herein may be implemented;

FIGS. 2A and 2B are diagrams of exemplary user devices of FIG. 1;

FIG. 3 is a is a diagram illustrating exemplary components of the user device of FIGS. 2A and 2B;

FIG. 4 is an exemplary functional block diagram of components implemented in a user device of FIG. 1;

FIG. 5 is an exemplary functional block diagram of components implemented in the service provider of FIG. 1;

FIG. 6 is illustrates a structure of an exemplary database for storing monitoring profile information received by monitoring profile logic of FIG. 5;

FIG. 7 is a flow diagram illustrating exemplary processing associated with wellness monitoring in the system of FIG. 1;

FIG. 8 is a flow diagram illustrating additional exemplary processing associated with wellness monitoring in the system of FIG. 1; and

FIG. 9 is a flow diagram illustrating still additional exemplary processing associated with wellness monitoring in the system of FIG. 1.

### DETAILED DESCRIPTION OF PREFERRED EMBODIMENTS

The following detailed description refers to the accompanying drawings. The same reference numbers in different drawings may identify the same or similar elements. Also, the following detailed description does not limit the embodiments disclosed herein.

Implementations described herein relate to devices, methods, and systems for facilitating the monitoring and exchanging of health and wellness related information. In some implementations, a mobile telephone or other portable electronic device may include components configured to monitor and/or determine information relating to the health and wellness conditions of a user. The health and wellness information may include health status condition information as well as other types of information, such as user's location. The device may transmit the health and wellness information to a service provider. A profile maintained at the service provider and associated with the device may include rules that identify actions to be executed in the event of various defined conditions relating to the health and wellness information. If designated by the defined rules, the service provider may initiate alerts, notifications, or calls to various individuals, entities, or devices.

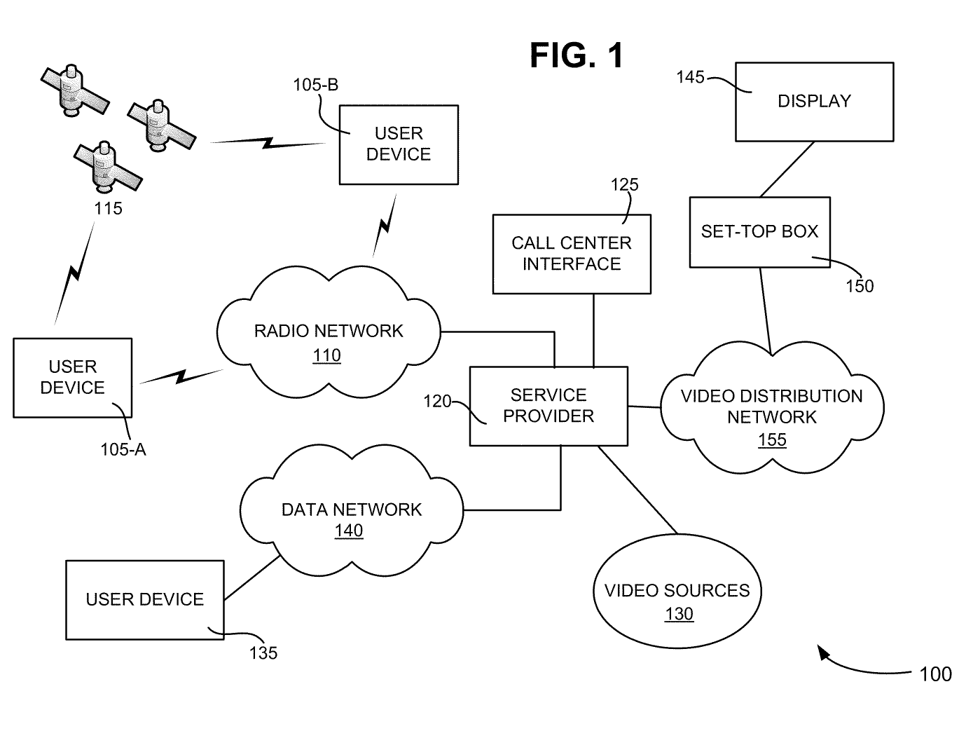

FIG. 1 is a block diagram of an exemplary system 100 in which systems and methods described herein may be implemented. As shown, system 100 may include a number of user devices 105-A and 105-B (collectively “user devices 105” and individually “user device 105) connected to a radio network 110 and one or more location determination devices 115. System 100 may also include a service provider 120 connected to radio network 110, a call center interface 125 connected to service provider 120, one or more video sources 130 operatively connected to service provider 120, user device 135 connected to service provider via data network 140, and a display 145 coupled to service provider 120 via a set-top box 150 and a video distribution network 155.

Consistent with embodiments described herein, user devices 105 may include any suitable device for enabling communication via radio network 110 and may include a cellular radiotelephone, personal digital assistant (PDA), pager with data communications and/or data processing capabilities, or any device capable of facilitating communication either with other user devices 105 or with service provider 120 via radio network 110. For example, user devices 105 may include a mobile telephone, PDA, gaming device, global positioning system (GPS)-capable device, or other portable device, such as a smart label or tag, embodiments of which are discussed below.

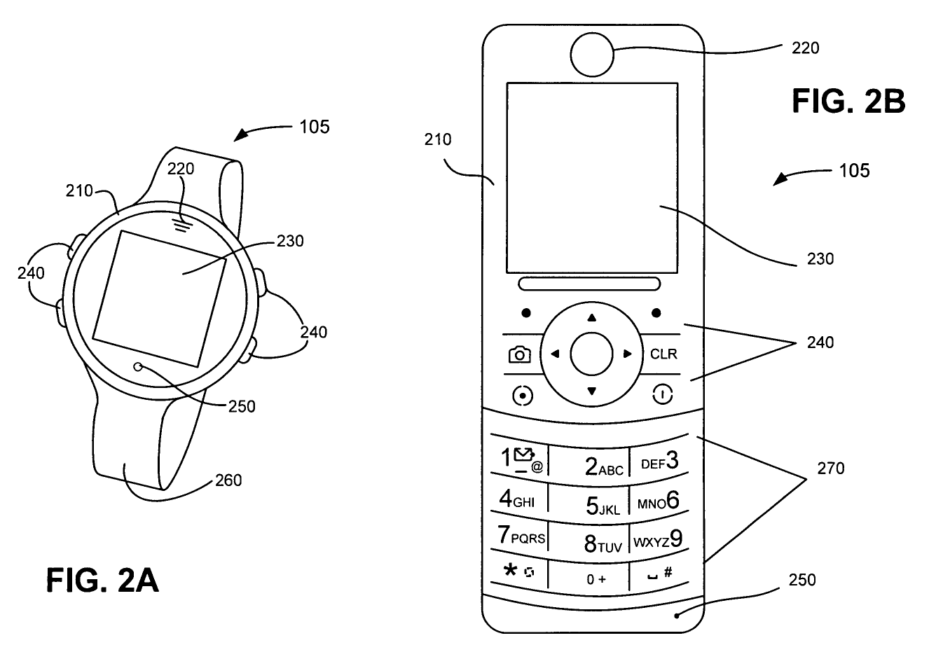

In an alternative implementation, one or more of user devices 105 may include a personal computer (PC), laptop computer, palmtop receiver, remote control device and/or any other appliance that may include a radiotelephone transceiver and applications for providing data processing and data communication functionality. As will be described in detail below with respect to FIG. 2A, user devices 105 may include communications devices configured to be worn by a user on an as-needed or continual basis. It should be understood that the two instances of user devices 105 illustrated in FIG. 1, are provided for simplicity. In practice, system 100 may include any number and type of user devices 105.

In addition, more than one user device 105 may be associated with a particular user. For example, a first user device 105 may correspond to a wearable device, second and third user devices 105 may correspond to motion sensors provided in the user's home, and fourth user device 105 may correspond to a piece of medical equipment, such as an insulin machine. In accordance with implementations described herein, each user device 105 may monitor wellness information relating to the user and may transmit the monitored information to service provider 120. In some implementations, one or more user devices 105 may transmit information to another user device, which in turn, may transmits the collected information to service provider 120.

Radio network 110 may include any suitable wireless network, such as, for example, a CDMA (Code-Division Multiple Access) network, a WCDMA (Wideband CDMA) network, a GSM (Global System for Mobile Communications) network, a GPRS (General Packet Radio Service) network, an EDGE (Enhanced Data Rates for GSM Evolution) network, an HSDPA (high speed downlink packet access) network, etc. In one embodiment, user devices 105 may communicate with other devices 105 or with service provider 120 using short-range wireless network standards, such as WiFi (e.g., IEEE 802.11x) or WiMAX (e.g., IEEE 802.16x). In yet other implementations, user device 105 may be configured to use multiple types of radio networks 110, depending on various factors, such as available signal strength, cost, etc. For example, user device 105 may be configured to communicate with service provider 120 via a cellular radio network (e.g., GSM/HSDPA) when user device 105 is out of range of a personal 802.11x wireless network. When in range, user device 105 may communicate with service provider 120 via the personal 802.11x wireless network.

Location determining devices 115 may include any combination of devices capable of enabling user devices 105 to determine the respective geospatial locations. Examples of location determining devices 115 may include GPS (global positioning system) satellites, cellular towers used in cell-tower triangulation, or WiFi (IEEE 802.11x) routers used in location-enabled WiFi services.

The GPS system is comprised of 27 GPS satellites (24 active and 3 backups) each configured to orbit the earth twice each day. The positions of the satellites are such that at least four GPS satellites are “visible” in the sky at any one time. Each satellite generates a radio signal that includes time and date, latitude, longitude, and satellite identification information. In order to accurately track a device location in two dimensions (e.g., no altitude or z-direction), signals from at least three satellites should be received, to determine a known location on the Earth's surface using a concept known as 3-D trilateration. In generally, trilateration works because the speed of the satellite signals and their respective locations are known. Accordingly, a time taken for a GPS receiver (e.g., user device 105) to “receive” a signal from each satellite may be used to identify the distance from the satellite to the receiver. Once distances from at least three satellites have been determined, the receiver's location may be determined, since there will be only one point on the Earth's surface that meets each of these distances. For more precise location identification including the receiver's altitude, a fourth satellite signal may be required. It should be understood that signals from more than four satellites may also be received at any one moment, thereby enhancing the performance of the GPS receivers. Additionally, the location of server devices 105 may be determined by using assisted global positioning system (aGPS), where the assistance data can include ephemeris data, approximate location, time, and other GPS aiding data needed to obtain location quickly or in obstructed line of sight locations (in building, wooded areas, etc.) from the satellites.

Cell tower-based and WiFi-based location services operate in a similar manner to the GPS system described above. However, instead of using signals streamed from satellites, these services use signals from terrestrial sources, such as cell towers and WiFi routers/access points. Given the known locations of these terrestrial signal sources, locations of connected devices may be ascertained with varying degrees of specificity. In an alternative implementation, determining locations of user devices 105 may involve using a network-based mobile station locator to track and store the geographic location of mobile stations over a given period of time. The stored geographic location information may be obtained from a telecommunications network (e.g., radio network 110 and service provider 120) instead of directly from user devices 105. Geographic location information may be obtained periodically (based on a predetermined time interval), continuously, or in an “on-demand” basis.

Service provider 120 may include a telecommunications service provider configured to receive and transmit various types of communication information, such as mobile telephone calls (e.g., via radio network 110), Internet Protocol packets (e.g., via data network 140), video (e.g., from video sources 130 and video distribution network 155), and circuit-based communications (e.g., public switched telephone network (PSTN) communications), etc. Although service provider 120 is depicted as a single block in FIG. 1, it should be understood that service provider 120 may include a number of entities, such as a PSTN central office, a video head end, a cellular telephone exchange, an Internet service provider, a data center, etc. In such implementations, the entities that together form service provider 120 may communicate with each other in a variety of ways, including packet or circuit-based communications networks, such as local area networks (LANs), wide area networks (WANs), etc.

Service provider 120 may include a wellness monitoring system configured to monitor and log information relating to user devices 105 and, by extension, users of user devices 105. Service provider 120's wellness monitoring system may enable guardians or caregivers associated with a user (e.g., a child or elderly individual) of a user device 105 to establish various triggers or rules to initiate defined event sequences (e.g., call actions, alerts, notifications, etc.) under specified conditions.

In addition, service provider 120 may provide notification service relating to the location or status of users associated with corresponding user devices 105. The notifications may be generated based on, for example, time-of-day and/or location of the user devices 105, a monitored health status of a user that is monitored by user devices 105, etc. For example, if a user device associated with or worn by a child is outside of a predetermined range from a defined geographic location (e.g., the child's school) between certain hours, the child's parents may be notified. As another example, lack of motion of an elderly user between defined waking hours may trigger a notification to be sent to the user's caregiver or medical/emergency personnel. In this manner, a subscriber, such as a parent, of the monitoring and notification service, can track the whereabouts of users (e.g., children) of user devices 105.

Call center interface 125 may include a combination of hardware and software components for enabling users or user devices 105 to interact with wellness monitoring systems or personnel. For example, call center interface 125 may include an interactive voice response system (IVR) for interacting with users via spoken prompts. Alternatively, call center interface 125 may include one or more phone banks for facilitating direct communication with live customer service personnel. As will be discussed in additional detail below, call center interface 125 may receive information regarding users of user devices 105. The information may be used in determining how to suitably respond to various monitored conditions.

In addition, call center interface 125 may provide customer service personnel with access to various features relating to user devices 105, such as navigational applications, message applications, service directories, etc. via service provider 120 and radio network 11O. As will be discussed below, service provider 120 may invoke call center interface 125 in a variety of circumstances, including those based upon monitored wellness data or upon calls from a user of user device 105. Because service provider 120 continually receives updated location and wellness information data from user devices 105, service provider 120 may forward such information to call center interface 125 for enhancing services provided by call center interface 125.

User device 135 may include any suitable device for enabling communication with service provider 120 via data network 140 and may include a mobile telephone, PDA, gaming device, personal computer (PC), laptop computer, palmtop receiver, netbook device, remote control device and/or any other appliance that may include a transceiver and other applications for providing data processing and data communication functionality. Data network 140 may include a packet-based data network, such as the Internet, a local area network (LAN), a wide area network (WAN), a metropolitan area network (MAN), a cellular network, a PSTN, a high-speed fiber optic network, (e.g., FiOS™), or any other network or a combination of networks.

As will be discussed in additional detail below, users of user device 135 may initiate, modify, monitor, and/or review wellness monitoring relating to user devices 105 via interactions with service provider 120 and/or call center interface 125. For example, service provider 120 may include a web server application reachable via data network 140. The web server application may host a web site configured to enable users of user device 135 to configure a wellness monitoring profile associated with a user device 105. In addition, various notifications and reporting functions of service provider 120 may be provided to users of user device 135 via data network 140.

Additionally, service provider 120 may be accessible by user devices (e.g., device 105) via a cellular gateway (not shown). In this manner, a mobile device (e.g., a user device 105) may upload (or download) monitoring profile information, monitoring report information, notifications, alerts, etc. to (or from) the service provider 120. As such, guardians or caregivers can input and modify a monitoring profile by manipulating the user device 105 itself or by configuring a monitoring profile on service provider 120 via user device 205. Further, in one implementation, both service provider 120 and user device 105 can be configured to automatically synchronize monitoring profile information when one or more changes are made, either on service provider 120 or on user device 105.

Video sources 130 may include television broadcast systems or other content providers from which video content may be received. Such video content may be selectively delivered to end users (e.g., display devices 145) via video distribution network 155 and/or set-top boxes 150. Video distribution network 155 may include various broadband access technologies, including, for example, digital subscriber line (DSL), fiber optic services (FiOS™), cable, worldwide interoperability for microwave access (WiMAX), etc., to connect set-top boxes 150 to the services offered by service provider 120. According to one embodiment, display 145 and set-top box 150, for example, may support high resolution video streams, such as data streams for high definition television (HDTV). In addition, set-top box 50 may encapsulate data into proper format with required credentials before transmitting the data onto video distribution network 155 and may de-encapsulate incoming traffic to dispatch data to display 145.

In an exemplary embodiment, display 145 may be configured to include Internet Protocol (IP) packet or other data processing capabilities (i.e., includes an Internet Protocol (IP) stack, or is otherwise network addressable), such that the functions of set-top box 150 are included in display 145. In such an implementation, display 145 may directly connect to video distribution network 155.

In one embodiment, service provider 120 may perform user authentication services to determine that users are indeed subscribers to received video information. A suitable authentication schema may require a username/password, a key access number, a unique machine or identifier of the user equipment (e.g., media access control (MAC) address), etc. Once the user equipment (e.g., set-top box 150) is authenticated, connections from set-top box 150 to service provider 120 can be established.

As will be discussed in additional detail below, call center interface 125 may cooperate with service provider 120 to receive and transmit various wellness monitoring-related communications. For example, in some implementations, service provider 120 may initiate a communication from call center interface 125 to a user device 105 (e.g., user device 105-A) upon the occurrence a predetermined triggering event. In other implementations, user device 105 may initiate the communication to call center interface.

In yet other implementations, call center interface 125 and/or service provider 120 may communicate with user device 135 and/or display device 145 to exchange information corresponding to the use of a user device 105 or to deliver alert, report, or notification information regarding monitored activities associated with user device 105.

FIGS. 2A and 2B are diagrams of exemplary user devices 105. Referring to FIG. 2A, user device 105 may include a wearable device that includes a housing 210, speaker 220, display 230, control buttons 240, microphone 250, and strap 260. Housing 210 may protect the components of user device 105 from outside elements. Speaker 220 may provide audible information to a user of user device 105. For example, speaker 220 may provide ringtones, beeping sounds or other sounds to alert the user to an event. For example, speaker 220 may be configured to output an alert relating to triggering event identified by user device 105, such as the user device 105 being outside of an allowed geographical area. Speaker 220 may also output audio information or instructions to a user of user device 105.

Display 230 may provide visual information to the user. For example, display 230 may include a liquid crystal display (LCD), a touch screen display or another type of display used to provide information to the user, such as information regarding a time of day, location information, health or wellness status information (e.g., pulse rate, blood pressure, blood sugar level, etc.), incoming or outgoing telephone calls, and/or incoming or outgoing electronic mail (email), instant messages (e.g., mobile instant messages (MIMs), short message service (SMS) messages, multi-media message service (MMS) messages, etc. Display 230 may also display information regarding various applications, such as a calendar application or text message application stored in user device 105, video games being played by a user, downloaded content (e.g., news or other information), etc.

Control buttons 240 may permit the user to interact with user device 105 to cause user device 105 to perform one or more operations, such as send communications (e.g., text messages or multi-media messages), place a telephone call, play various media, etc. For example, control buttons 240 may include a send button, an answer button, a dial button, a hang up button, a clear button, a play button, etc. In an exemplary implementation, control buttons 240 may also include one or more buttons that may be used to launch an application program, such as a messaging program. Further, one of control buttons 240 may be a menu button that permits the user to view options associated with executing various application programs, such as a social interaction program, stored in user device 105. In some implementations, functions associated with control buttons 240 may be duplicated or replaced by other interactive control elements, such as a touch screen display 230 or a voice response system. Microphone 250 may receive audible information from the user, such as telephone communications and/or voice input. Strap 260 may include any mechanism for securely affixing user device 105 to the user or the user's clothing. In some implementations, strap 260 may be configured to lockingly attach to a user, so that it may not be easily removed, or that removal of user device 105 may trigger a notification to a caregiver or other entity.

As illustrated, the embodiment of FIG. 2A provides user device 105 in one of a variety of user-wearable form factors. More specifically, user device 105 of FIG. 2A illustrates a wristwatch, designed to be worn on the wrist of a user. Although not illustrated, other exemplary wearable form factors for user device 105 may include a pendant style device configured for wearing via a chain or lanyard, a brooch or other pin-on or clip-on on style device, a ring, etc. Furthermore, the elements of device 105 may be incorporated into other user devices, such as a wireless headset, etc. Such wearable form factors may facilitate increased reliability of the wellness monitoring system by making it easy for a user to maintain possession of user device 105.

Referring to FIG. 2B, user device 105 may include housing 210, speaker 220, display 230, control buttons 240, microphone 250, and keypad 260. Components 210-250 for user device 105 in FIG. 2B may provide similar functionalities as the corresponding components in FIG. 2A.

Keypad 270 may include a telephone keypad. As illustrated, many of the keys on keypad 270 may include numeric values and various letters. For example, the key with the number 2 includes the letters A, B and C. These letters may be selected by a user when inputting text to user device 105. Other keys on keypad 250 may include symbols, such as the plus symbol (i.e., +), the minus symbol (i.e., −), the at symbol (i.e., @), etc. These symbols may be used to perform various functions.

The embodiment of FIG. 2B illustrates a non-wearable form, such as a portable or mobile telephone device. Other exemplary non-wearable forms for user device 105 in accordance with embodiments described herein may include PDAs, hand-held gaming devices, personal navigation system devices, or netbook, laptop, notebook, or ultra mobile personal computers (UMPCs), etc.

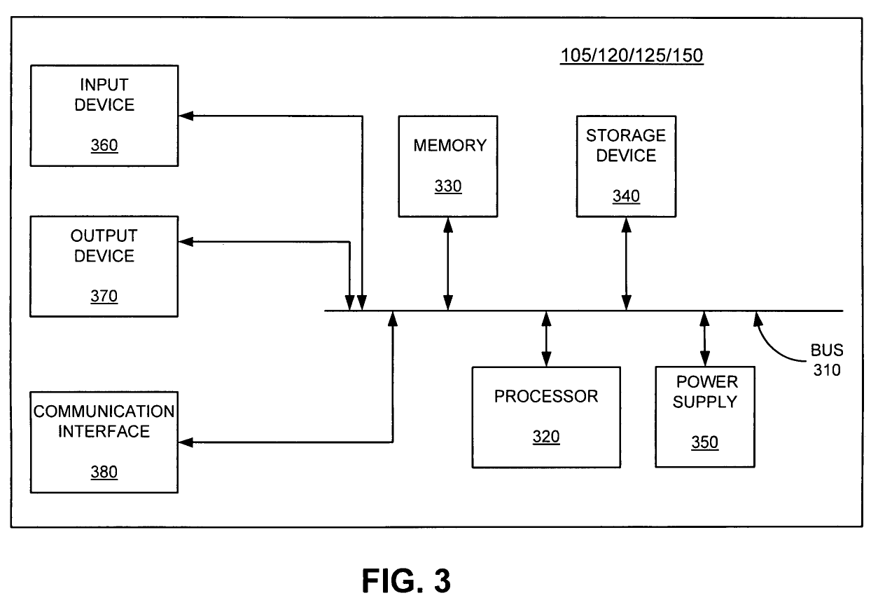

FIG. 3 is a diagram illustrating components of user devices 105. In some implementations, user device 135, set-top box 150, and/or devices in service provider 120 and call center interface 125 may include similar components. Referring to FIG. 3, user device 105 may include bus 310, processor 320, memory 330, storage device 340, power supply 350, input device 360, output device 370, and communication interface 380. It should be understood that user device 105 may be configured in a number of other ways and may include other or different elements. For example, user device 105 may include one or more modulators, demodulators, encoders, decoders, etc., for processing data.

Bus 310 may include a path that permits communication among the elements of user device 105. Processor 320 may include one or more processors, microprocessors, application specific integrated circuits (ASICs), field programmable gate arrays (FPGAs), or other processing logic that may interpret and execute instructions. Memory 330 may include a random access memory (RAM) or another type of dynamic or static (e.g., read only memory (ROM)) storage device that may store information and instructions for execution by processor 320. Storage device 340 may include a magnetic and/or optical recording medium and its corresponding drive. Power supply 350 may include a battery or other power source used to power user device 105.

Input device 360 may include one or more mechanisms that permit a user to input information to user device 105, such as control keys 240, microphone 250, keypad 270, a touch screen, such as display 230, a mouse, a pen, voice recognition and/or biometric mechanisms, such as a pulse monitor, glucose monitor, fingerprint scanner, etc.

Output device 370 may include one or more mechanisms that output information to the user, including a display, such as display 230, a printer, one or more speakers, such as speaker 220, a vibrating mechanism that provides haptic feedback to a user, etc.

Communication interface 380 may include any transceiver-like mechanism that enables user device 105 to communicate with other devices and/or systems. For example, communication interface 380 may include mechanisms for communicating via a network, such as a wireless network. In these implementations, communication interface 380 may include one or more radio frequency (RF) transmitters, receivers and/or transceivers and one or more antennas for transmitting and receiving RF data via a network. In addition, as briefly described above, communication interface 380 may include location determining logic, such as GPS or assisted GPS logic, for receiving and/or determining location-related information corresponding to user device 105 using location determining devices 115 (e.g., GPS satellites).

Communication interface 380 may also include an infrared (IR) transmitter and receiver and/or transceiver that enable user device 105 to communicate with other devices via infrared (IR) signals. Communication interface 380 may also include a modem or an Ethernet interface to a LAN or other network for communicating with other devices in system 100. In yet other implementations, communication interface 380 may be configured use one or more short-range wireless technologies, such as radio frequency identifiers (RFID), Bluetooth®, or near field communication (NFC). Such technologies may provide for the exchange of data between devices in close physical proximity to each other. Communication interface 380 may include other mechanisms for communicating with other devices 105 or via a network.

Such a network may include one or more wired, wireless and/or optical networks that are capable of receiving and transmitting data, voice and/or video signals, including multimedia signals that include voice, data and video information. For example, a network suitable for use with user device 105 may include one or more public switched telephone networks (PSTNs) or other type of switched network. The network may also include one or more wireless networks and may include a number of transmission towers for receiving wireless signals and forwarding the wireless signals toward the intended destination. The network may further include one or more packet switched networks, such as an Internet protocol (IP) based network, a local area network (LAN), a wide area network (WAN), a personal area network (PAN), an intranet, the Internet, or another type of network that is capable of transmitting data.

In some implementations consistent with embodiments described herein, user device 105 may provide a wellness monitoring platform for enabling wellness monitoring system users to actively monitor the wellness of various individuals. In some implementations, user device 105 may also enable a user to receive wellness related alerts or notifications, make and receive telephone calls, send and receive electronic mail, text messages, instant messages (IMs), mobile IMs (MIMs), short message service (SMS) messages, etc., and execute various other applications. User device 105, as described in detail below, may also perform processing associated with determining a geographic location of user device 105 and wellness related information, and forwarding location information and the wellness-related information to service provider 120 for use in executing wellness related rules corresponding to the user device 105.

Service provider 120 and call center interface 125, as described in detail below, may perform processing associated with receiving and storing profile information corresponding to user devices 105, receiving wellness related information from user devices 105, and initiating event sequences (e.g., call actions, alerts, notifications, etc.), which are identified in the profile information, based on the received wellness related information. In addition, service provider 120 and call center interface 125 may perform processing associated with receiving requests for information corresponding to the received wellness related information.

User device 105, service provider 120, and call center interface 125 may perform these operations in response to their respective processors 320 executing sequences of instructions contained in a computer-readable medium, such as memory 330. A computer-readable medium may be defined as a physical or logical memory device. The software instructions may be read into memory 330 from another computer-readable medium, such as data storage device 340, or from another device via communication interface 380. The software instructions contained in memory 330 may cause processor 320 to perform processes that will be described later. Alternatively, hard-wired circuitry may be used in place of or in combination with software instructions to implement processes consistent with the embodiments described herein. Thus, implementations described herein are not limited to any specific combination of hardware circuitry and software.

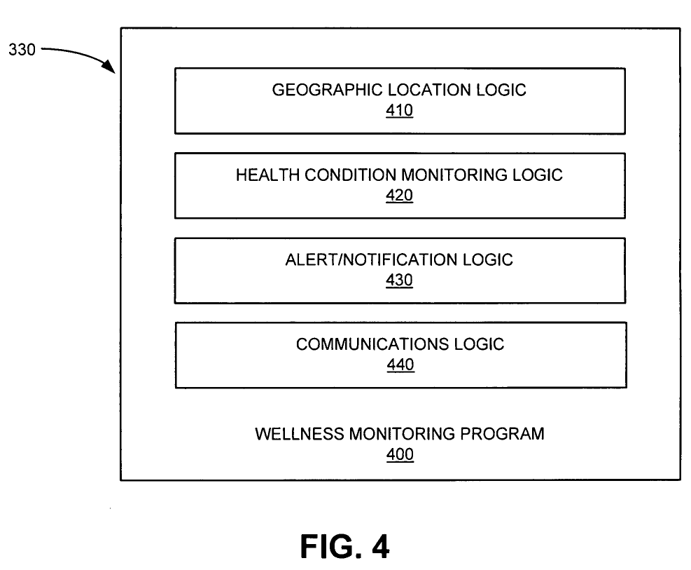

FIG. 4 is an exemplary functional block diagram of components implemented in user device 105 of FIGS. 2A and 2B. The logical blocks illustrated in FIG. 4 may be implemented in software, hardware, a combination of hardware and software.

Referring to FIG. 4, memory 330 may include a wellness monitoring program 400 executable by processor 320. As will be discussed in detail below, wellness monitoring program 400 may be configured to enable wellness monitoring system users to monitor and track wellness information corresponding to a user of user device 105, such as geographic location, call history, motion activity, health information, etc. As illustrated, wellness monitoring program 400 may include geographic location logic 410, health condition monitoring logic 420, alert/notification logic 430, and communications logic 440. Wellness monitoring program 400 and its various logic components are shown in FIG. 4 as being included in user device 105. In alternative implementations, these components or a portion of these components may be located externally with respect to user device 105. For example, in some implementations, one or more of the components of wellness monitoring program 400 may be located in or executed by a remote network device.

Geographic location logic 410 may include logic configured to dynamically determine a geographic location of user device 105. For example, geographic location logic 410 may use information received from location determining devices 115 (e.g., GPS satellites, etc.) to periodically and automatically determine its geographic location. The determined location may be stored, e.g., in storage 340 of device 105, and may also be periodically transmitted to service provider 120 via radio network 110.

Health condition monitoring logic 420 may include logic configured to monitor user device 105 and to store health condition information corresponding to a user of user device 105. For example, health condition monitoring logic 420 may be configured to periodically query the user via display 230 and/or speaker 220 to input health-related information, such as confirmation that medicine has been taken, meals eaten, etc. Alternatively, health condition monitoring logic 420 may be configured to automatically and periodically monitor various health conditions of the user, such as pulse rate, blood pressure, activity levels (e.g., using an accelerometer, etc.). The received and/or monitored health condition information may be stored, e.g., in storage device 340 of device 105, and may also be periodically transmitted to service provider 120 via radio network 110.

Alert/notification logic 430 may be configured to output audio and/or visual alerts or notifications (e.g., via display 230 and/or speaker 220) in response to the identified location (as identified by location monitoring logic 410) and/or health information (as identified by health condition monitoring logic 420), or in response to received communications from service provider 120, e.g., calls from call center interface 125.

Communications logic 440 may include logic configured to transmit or receive information from service provider 120 via communication interface 380. As described briefly above, communications logic 440 may include any suitable transceiver mechanism for communicating via radio network 11O. In one implementation, communications logic 440 may be configured to periodically transmit geographic location, health information, and time stamp (e.g., date and time) information to service provider 120 via radio network 110.

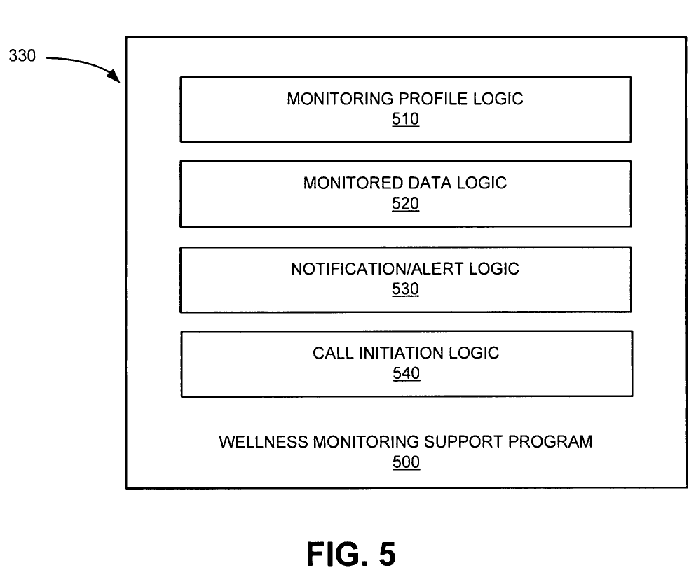

FIG. 5 is an exemplary functional block diagram of components implemented in service provider 120 of FIG. 1, such as by processor 320 executing a program stored in memory 330 (e.g., in one or more server or database devices (not shown) associated with service provider 120). The logical blocks illustrated in FIG. 5 may be implemented in software, hardware, or a combination of hardware and software.

Referring to FIG. 5, memory 330 may include a wellness monitoring support program 500 executable by processor 320. As will be discussed in detail below, wellness monitoring support program 500 may be configured to enable wellness monitoring system users to establish monitoring profiles corresponding to each user device 105. Once a monitoring profile has been established, wellness monitoring support program 500 may be configured to enable system users to monitor geographic location, call history, motion activity, health information, etc. associated with user devices 105 based on the established profiles. In addition, provisions in the established monitoring profile may define various sequences of communications and notification events as well as the criteria that may be used to trigger the defined sequences of communications and notification events.

As illustrated, wellness monitoring support program 500 may include monitoring profile logic 510, monitored data logic 520, notification/alert logic 530, and call initiation logic 540. Wellness monitoring support program 500 and its various logic components are shown in FIG. 5 as being included in service provider 120. In alternative implementations, these components or a portion of these components may be located in a number of devices associated with service provider 120, such as distributed server devices, databases, and call center devices.

Monitoring profile logic 510 may include logic configured to receive information corresponding to wellness conditions monitored at user device 105 (or group of user devices). For example, monitoring profile logic 510 may be configured to receive monitoring profile information from one or more users of the wellness monitoring system, such as a caregiver, guardian, parent, etc., associated with the user of user device 150. Exemplary monitoring profile information may include rules that define the circumstances in which various actions are performed.

In some implementations, monitoring profile information may be explicitly received from a user, such as a caregiver. For example, monitoring profile logic 510 may provide an interface (e.g., a web server, interactive voice response system, call center, etc.) for receiving monitoring profile information from the user. Such an interface may query the user for monitoring profile information based on information that has been previously submitted. For example, a web-based implementation may have an initial set of questions regarding the monitoring profile to be set up, such as the age of the person being monitored, etc. Follow-up questions may then be presented that are tailored based on responses to the initial set of questions.

In another implementation, monitoring profile logic 510 may receive monitoring profile information via selection of one of a number of pre-set monitoring profiles that are presented to the user for selection. For example, monitoring profile logic 510 may provide users with a listing of pre-set monitoring profiles to generally meet identifiable monitoring scenarios, such as a child safety profile, an elderly health emergency profile, an elderly location monitoring profile, a vacation monitoring profile, a work week monitoring profile, etc. Upon selection of one of the pre-set monitoring profiles, monitoring profile logic 510 may query users with follow-up questions directed to completing the specifics of the profile (e.g., notification contacts, etc.).

In some implementations, the content of the available pre-set monitoring profiles may be based on collaborative information based in part on profiles established by other users of the wellness monitoring system. For example, assume that a number of users have defined monitoring profiles that include a rule to notify a pharmacy of an upcoming medication need. Based on these previously established monitoring profiles, monitoring profile logic 510 may include this practice in subsequent pre-set monitoring profiles, or, alternatively, may query a user for inclusion of a similar type of rule into an established monitoring profile.

In yet another implementation, monitoring profile logic 510 may include intelligence configured to identify patterns and/or changes in activity or behavior (i.e., of a user of user device 105) that may warrant additions or modifications to an existing monitoring profile. For example, monitoring profile logic 510 may apply pattern matching based on the habits of the user to create or suggest new rules for inclusion in a monitoring profile. For instance, a user and/or their family or caregiver may establish initial monitoring rules based on certain activities, geographic locations, or what actions they initially believe important or relevant. However, changes in the user's life—such as long term geographic changes, etc. that are not explicitly incorporated into a monitoring profile, may be recognized by service provider 120. Monitoring profile logic 510 may query the user relating to such recognized changes, potentially resulting in modifications to the established monitoring profile. An exemplary pattern that may trigger the above-described pattern matching may include consistent geographic changes, however, it should be understood that any identifiable change may be used to trigger a rule modification/additional query. For example, recognized late night wandering due to early onset of dementia that was not anticipated by the family may trigger an inquiry to the caregiver or user to include one or more monitoring rules relating to dementia-related notifications or emergency response.

As will be discussed in additional detail below, exemplary monitoring rules may define combinations of conditions and event sequences that are to take place upon occurrence of the defined combination of conditions. In some implementations, the event sequences may include notification and/or alerting events corresponding to the monitored conditions. In one embodiment, sets of defined conditions may result in different event sequences based on changes to other related conditions. For example, a monitoring rule may indicate that user device 105 outside of a predefined geographic boundary in or around a users home may be result in different event sequences than user device 105 exceeding a predefined geographic boundary around a different geographic region, such as a vacation destination.

In one implementation, monitoring profile logic 510 may receive the monitoring profile information from, for example, user device 135 via data network 140 or other user devices 105 via radio network 110. In such an implementation, monitoring profile logic 510 may include a web server application (not shown) configured to provide an interactive web site for receiving monitoring profile information. The received monitoring profile information may be stored as a monitoring profile in a database structure, e.g., storage device 340 in service provider 120.

Monitored data logic 520 may include logic configured to receive location information, monitored health condition information, and timestamp information from user devices 105. In some implementations, geographic location logic 520 may be configured to receive unsolicited information from user devices 105; i.e., user devices 105 may “push” their geographic location, health condition information, and timestamp information to monitored data logic 520, for example, on a periodic basis via radio network 110. In other implementations, monitored data logic 520 may periodically query or request updated location and health condition information from user device 110 via radio network 110. As briefly described above, multiple user devices 105 may be configured to monitor and provide wellness related information to service provider 120, either individually, or collectively.

Received geographic location and health condition information associated with user devices 105 may be stored along with the corresponding timestamp information, for example, in a database associated with wellness monitoring support program 500. In one implementation, monitored data logic 520 may include a web server application (not shown) configured to provide an interactive web site for enabling users of wellness monitoring system to view the received location and health condition information for a designated period of time, or alternatively, in real time. For example, geographic location logic 520 may provide an interface depicting a map overlaid with a path corresponding to the received geographic location information. At points corresponding to each data retrieval period, corresponding health condition information may be provided or selected via the interface.

Notification/alert logic 530 may include logic configured to compare the geographic location and health condition information received by monitored data logic 520 to the monitoring profile information established by monitoring profile logic 510. Notification/alert logic 530 may identify any received information that meets any conditions or rules, as set forth in a monitoring profile, that may result in notifications or alerts. When a rule requires a notification or alert, notification/alert logic 530 may identify a party or individual to notify based on the monitoring profile and the identified condition/location information. Notification/alert logic 530 may then initiate the required notification or alert. For example, notification/alert logic 530 may transmit an email or SMS message, may initiate a call via call initiation logic 540, may transmit an alert or notification to display 145 via set-top box 150, etc. In one implementation, transmitted notifications/alerts may include information associated with user device 105, such as identification information, call-back information, location information, etc. In addition, transmitted notifications/alerts may include all or parts of the received geographic location and health condition information, in accordance with the rule.

In one implementation consistent with embodiments described herein, devices configured to receive alerts and notifications from notification/alert logic 530 (e.g., user devices 135 (mobile phones, PDA's, GPS systems, etc.), set-top box 150, other user devices 105, etc.) (generally referred to herein as “notification receiving devices”) may be configured to respond to various notifications from notification/alert logic 530. For example, notification receiving devices may be configured to provide an indication of a user's (of the notification receiving device) “status” in relation to a particular monitoring profile.

For example, assume that a monitoring profile has been established that includes rules that result in various notification receiving devices being notified or alerted in response to monitored conditions, such as emergency calls placed through a user device 105, a geographic location of user device 105, etc. In one implementation, each notification receiving device may be configured to provide a indication (e.g., a graphical or audible indication) that indicates that the notification receiving device has been designated as “on duty” or “active.” For example, assume that a monitoring profile has been established that defines notification receiving device A as being notified in the event of an emergency condition daily from 8:00 AM to 7:59:59 PM and notification receiving device B as being notified in the event of an emergency condition daily from 8:00 PM to 7:59:59 AM.

Based on this profile, alert/notification logic 530 may, in one implementation, transmit an “on duty” message to receiving notification device A at 8:00 AM notifying receiving device A of its “on duty” status and the duration of the duty interval. Upon receiving the “on duty” notification from alert/notification logic 530, notification receiving device A may provide an indication to the user of notification receiving device A. In one embodiment, the indication may include an indicator light (e.g., a LED, etc.), an icon or other graphical element provided on a user interface associated with notification receiving device A.

In another implementation, the “on duty” message from alter/notification logic 530 may be transmitted continually in periodic intervals throughout the duty period, and may include real-time monitoring information associated with the monitored user device 105. For example, the “on duty” message from alter/notification logic 530 may include geographic location information associated with user device 105. This information may enable the user of receiving device A to easily monitor the geographic location of user device 105 during the time period in which they are on duty. On in exemplary implementation, notification receiving device A may be a mobile phone or GPS system that, when “on duty” continually displays (or can display) the geographic location of user device 105, but which, when not “on duty” does not provide this information. In yet another implementation, notification receiving device A may include a set-top box 150 configured to display a graphical representation of the “on duty” status.

In other implementations, notification receiving devices may be associated with emergency services or medical personnel associated with user device 105. For example, an interface (e.g., at user device 135, etc.) associated with a medical personnel may be configured to receive “on duty” messages from alert/notification logic 530 informing the notification receiving devices that they are on duty with respect to user device 105. Such an implementation may be particularly useful for medical personnel or emergency service providers attempting to determine staffing needs, etc.

In addition to time of the day or day of the week based on duty messages, alert/notification logic 530 may be configured to transmit on duty messages based on other conditions, such as a geographic location of user device 105. For example, the monitoring profile may define notification receiving device A as being notified when user device 105 is in (or around) a first geographic location (e.g., home) and notification receiving device B as being notified when user device 105 is in (or around) a second geographic location (e.g., vacation house).

Call initiation logic 540 may include logic configured to initiate a telephone call to call center interface 125 or other telephone devices/systems, e.g., via a conventional PSTN or via data network 140. Call center interface 125 may be staffed with customer service personnel, emergency service personnel, or medical personnel to handle the call. In some implementations, call initiation logic 540 may automatically place a call to a caregiver or emergency services personnel or other notification receiving devices, depending on the monitored conditions. In other implementations, call initiation logic 540 may initiate a call based on a communication received from user device 105, such as a request to communicate with call center interface 125 or with emergency services personnel. Depending on the manner in which call is initiated, different information may be forwarded with the call, such as user identification information, location information, and/or health status information.

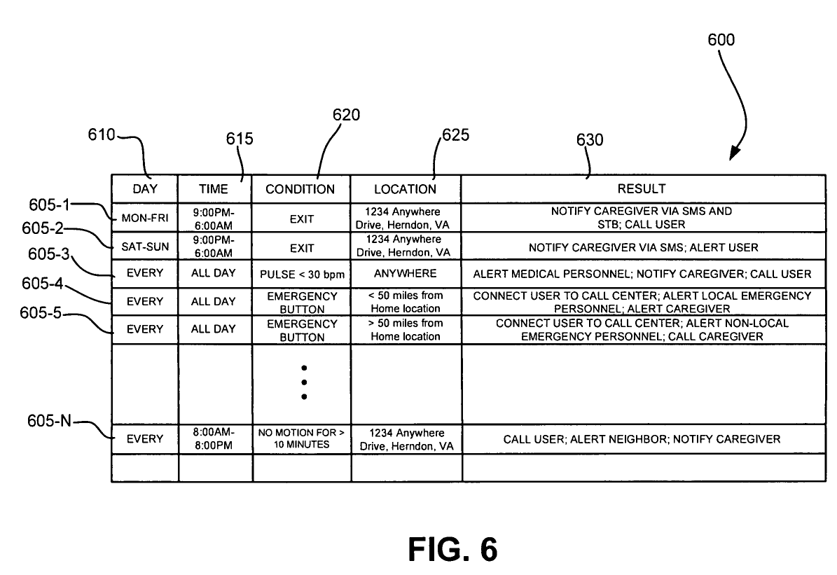

FIG. 6 illustrates a structure of an exemplary database 600 for storing monitoring profile information received by monitoring profile logic 510. Referring to FIG. 6, database 600 may include a number of monitoring entries 605-1 to 605-N (collectively “entries 605” or individually “entry 605”). Each entry 605 corresponds to a monitoring rule in the monitoring profile associated with a particular user device 105. Although only a single profile is depicted in database 600 it should be understood that database 600 would typically include profiles corresponding to each user device 105.

Each monitoring entry 605 may include a day field 610, a time field 615, a condition field 620, a location field 625, and a result field 630. As described above, monitoring profile logic 510 may receive monitoring profile information from caregivers or users associated with user devices 105. Additionally, each field 610-630 in entries 605 may include a plurality of fields. For example, result field 630 may include a plurality of fields indicating a number of actions to be taken upon satisfaction of the defined rule criteria, e.g., multiple notifications, call initiations, etc.

Day field 610 may list a day or days of the week on which the rule is to be applied. For example, entry 605-1 indicates MON-FRI in day field 610. This indicates that the rule provided in entry 605-1 is to be applied on weekdays, but not on a weekend. Time field 615 may indicate a time or time range during which the rule is to be applied. For example, entry 605-1 indicates 9:00 PM to 6:00 AM in time field 615. This indicates that the rule provided in entry 605-1 is to be applied during nighttime.

Condition field 620 may indicate conditions upon which actions specified in result field 630 are to be performed. The conditions may be related to the health condition (e.g., heart rate, blood pressure, etc.), geographic location, placement of an emergency call from user device 105, etc.

In one implementation, condition field 620 may include terms that specify conditions (e.g., exit, entry, leaving, entering, etc.) that may be satisfied when the user moves relative to a default distance (e.g., 100 or 200 feet) from a geographic location. For example, condition field 620 of field 605-1 includes “Exit,” which may be satisfied when user device 105 travels more than a particular distance from a geographic location.

Some conditions may not be related to user locations or user proximity. Such non-proximity conditions may include health conditions, such as activity level, heart rate, blood pressure, pacemaker battery status, emergency call requests, etc. Other conditions may include time of day or day of week for providing reminders or status reports and non-emergency call center requests. As described in additional detail below, monitoring profile rules relating to such non-proximity conditions may include location-related elements, in order to enhance the effectiveness of the system.

Location field 625 may include location information associated with the condition. Location field 625 includes an indication of a location associated with the condition. Although certain conditions relate to or require location information to be satisfied, other conditions, such as health status conditions may be invoked irrespective of a geographic location of the user device. Entries relating to such conditions may not designate a location in location filed 625. As illustrated in FIG. 6, location field 625 of entry 605-1 indicates “1234 Anywhere Drive, Hemdon, Va. 20170.” As described above, satisfaction of proximity-related conditions may be based on a determined location of user device 105 relative to the location provided in location field 625. For entry 605-1, the “Exit” condition may be satisfied when user device 105 travels more than the defined distance from 1234 Anywhere Drive, Herndon, Va. 20170, as provided in location field 625.

As discussed above, for non-proximity related conditions, such as health status conditions, received location information that matches the information in location field 625 may be used to determine what result action to perform, such as what healthcare or emergency services personnel to contact, what notifications or alerts to transmit, etc.

Result field 630 may indicate one or more actions to perform upon satisfaction of rule elements, i.e., the day, time, condition, and location information. For example, as illustrated in FIG. 6, result field 630 of entry 605-1 indicates the following actions: “notify caregiver via SMS” and “initiate call between call center interface 125 and user device 105.” Upon satisfaction of the rule elements provided in monitoring entry 605-1, service provider 120 may perform the actions.

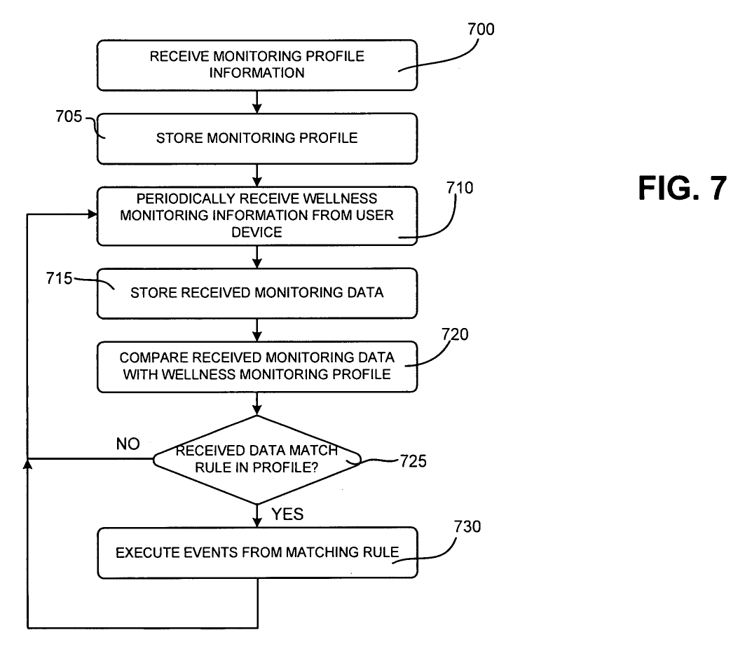

FIG. 7 is a flow diagram illustrating exemplary processing associated with wellness monitoring in system 100. Processing may begin with service provider 120 (e.g., monitoring profile logic 510) receiving monitoring profile information (block 700). For example, monitoring profile logic 510 may receive monitoring rules including day, time, condition, location information, and result information corresponding to an identified user device 105. As described above, monitoring profile logic 510 may receive this information from a user of the monitored user device 510, a user of another device, such as user device 135, such as a caregiver or guardian. In some implementations, the monitoring profile information may be received and/or modified via a web interface provided by service provider 120. Further, as described above, monitoring profile logic 510 may be configured to identify and suggest suitable rules and profile elements, based on pattern recognition with respect to user device 105, or based on collaborative best practices information identified based on collective numbers of monitoring profiles. Such suggestions may be identified and presented either during initial profile creation or subsequent to profile creation.

In addition, as described above, the received monitoring profile information may establish rules that include different result event sequences based on different triggering conditions. For example, certain types of medical-related emergencies may result in call initiation to emergency services personnel, yet other location or health status information may result in transmission of notifications to caregivers or to the user of user device 105. Similarly, an identical monitored condition may have different event sequences based on the location information associated with user device 105 or based on time or date information.

Service provider 120 may store a monitoring profile based on the received monitoring profile information (block 705). For example, monitoring profile logic 510 may store the received monitoring profile information in a database or other storage structure for subsequent retrieval during wellness monitoring operations, such as reporting or reviewing monitoring information.

In one implementation consistent with embodiments described herein, various individuals may establish, review, and/or modify a profile associated with a user device 105. For example, a user of user device 105 may be able to review, create, or modify profile rules or conditions in some implementations. For example, an independent, elderly user who participates in wellness monitoring system 100 may tailor her profile to ensure that, should a medical emergency or other unforeseen event occur, the user would be able to easily contact or communicate with those capable of assisting the user (e.g., medical services personnel, loved ones, etc.). In another example, a caregiver or guardian (e.g., parent) associated with the user of user device 105 may be able to establish, review, and/or modify a profile associated with a user device 105. In some unplementations, user of user device 105 may be unable to establish, modify, or review the monitoring profile.

Service provider 120 may periodically receive wellness monitoring information from monitored user device 105 (block 710). For example, as described above, monitored data logic 520 may periodically receive location information, monitored health information, and timestamp (e.g., date/time) information from user device 105. In some implementations, service provider 120 may request the information from user device 105, while in other implementations, user device 105 may automatically transmit the information to service provider 120, via, for example, radio network 110. The received monitoring data may be stored in a database or other data structure associated with service provider 120 (block 715).

Service provider 120 may compare the received location, monitored health, and timestamp information with the rules established in the wellness monitoring profile (block 720). For example, notification/alert logic 530 may compare the received location, monitored health, and timestamp information with the rules established in the wellness monitoring profile. Service provider 120 may determine whether the received monitoring information matches any rules established in the monitoring profile (block 725). For example, notification/alert logic 530 may determine whether the received monitoring information matches day, time, condition, and location information corresponding to a rule in the monitoring profile. If so (block 725—YES), service provider 120 may execute the sequence of events identified in the applicable rule (block 730). For example, assume notification/alert logic 530 identifies the actions or events provided in result field 630 of the matching rule (e.g., an entry 605 in database 600). If notification/alert logic 530 determines that the received monitoring information does not match day, time, condition, and location information corresponding to a rule in the monitoring profile (block 725—NO), the process returns to block 710 for a next monitoring interval. Any suitable monitoring interval (e.g., 1 minute) may be used based on various factors such as criticality of the information being monitored, battery limitations, cost, etc.

In one implementation, a communication initiated by user device 105, such as a call request or emergency notification request, may trigger immediate execution of blocks 710-725.

As discussed above, the actions or events provided in result field 630 may include notifications, alerts, call initiations, etc. The notifications and alerts may be formatted and transmitted via any suitable communication media to user device 135 via data network 140, to display 145 via set-top box 150 and video distribution network 155, etc. Accordingly, service provider 120 may include one or more gateway interfaces (not shown) for facilitating communications across disparate communication formats. Moreover, as described above, alert/notification logic 530 may be configured to transmit on duty messages to notification receiving devices upon occurrence of on duty conditions, as identified in the monitoring profile. Upon receipt of the on duty messages, a receiving device may display a graphic or audible indication of the on duty status of the receiving device.

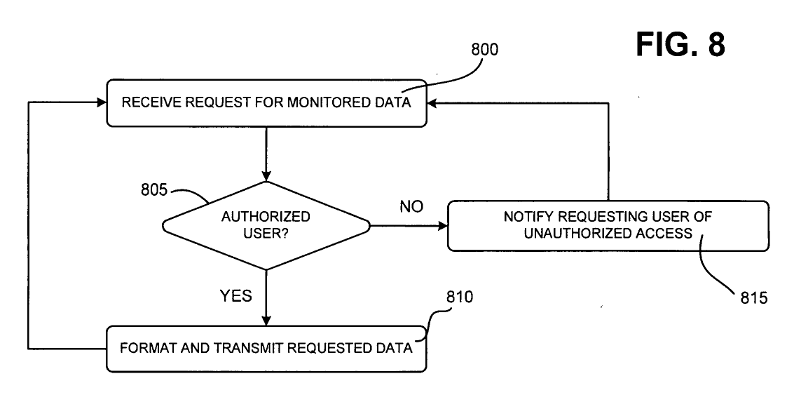

FIG. 8 is a flow diagram illustrating additional exemplary processing associated with wellness monitoring in system 100. Processing may begin with service provider 120 (e.g., monitored data logic 520) receiving a request for monitored data associated with a user device 105 (block 800). In some implementations, monitored data logic 520 may include a web server application configured to provide a web interface for facilitating receipt of such a request. In one implementation, the received request may be formatted as a query. Alternatively, the received request may designate predefined sets of data. Service provider 120 may determine whether the request is associated with an authorized user (block 805). For example, the received request may include or may be otherwise associated with login information associated with the user requesting the data. Alternatively, service provider 120 may request such login information upon receipt of the request. Regardless, monitored data logic 520 may determine whether the received login information is accurate and, if so, whether the user associated with the login information is authorized to receive the requested monitored data.

In one embodiment, an established monitoring profile may designate access privileges associated users or groups of users. Different users/groups may have access to different information. For example, some users, such as healthcare providers (e.g., doctors, nurses, physical therapists, etc.) may have access to health monitoring information, such as blood pressure measurements, pulse measurements, etc. However, these individuals may not have access to historical geographic location information or call histories. Conversely, a guardian or caregiver may have access to all of the stored information.

If it is determined that the request is associated with an authorized user (block 805—YES), service provider 120 may format the requested data and transmit the data to the requesting user (block 810). In some implementations, the requested data may be formatted based on the communication medium or device associated with the request. For example, data responsive to a request received via set-top box 150 may be formatted in a manner different from data responsive to a request received via mobile user device 105. If it is determined that the request is not associated with an authorized user (block 805—NO) (e.g., that login information is incorrect or that the user does not have access to the requested data), service provider 120 may notify the requesting user regarding the lack of authorization (block 815) and processing may return to block 800.

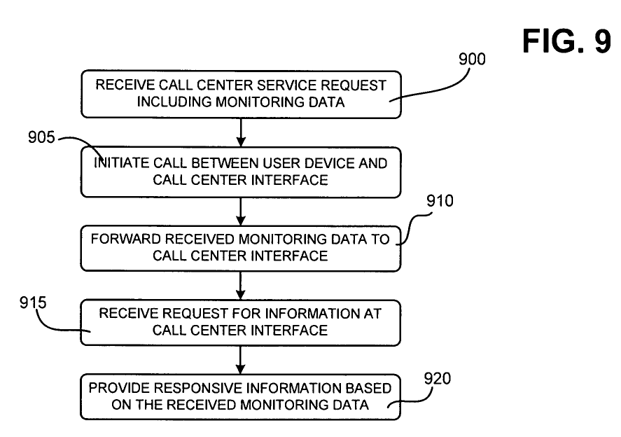

FIG. 9 is a flow diagram illustrating still additional exemplary processing associated with wellness monitoring in system 100. Processing may begin with service provider 120 receiving a call center service request from user device 105 (block 900). The call center service request may include monitoring data collected by user device 105, such as geographic location information, timestamp information, and health status information. Service provider 120 may initiate a call between user device 105 and call center interface 125 based on the request (block 905). Service provider 120 may forward the received monitoring data to call center interface 125 (block 910).

During the call, call center interface 125 (e.g., through an IVR system or customer support personnel) may receive a request for information from user device 105 (block 915). Call center interface 125 may, based on the received request and the monitoring data, provide responsive information to user device 105 (block 920).

By providing for the collection and forwarding of location and health condition information at the time at which a call center call is made, systems consistent with embodiments described herein may provide more timely and accurate information to user.

Implementations described herein relate to devices, methods, and systems for facilitating the monitoring and exchanging of health and wellness related information. In some implementations, a mobile telephone or other portable electronics device may include components configured to monitor and/or determine information relating to the health and wellness of a user, such as geographic location and health status condition information. Logic associated with the device may be configured to transmit the health and wellness information to a service provider. A profile established at the service provider and associated with the portable device may include rules that identify actions to be executed in the event of various defined conditions relating to the collected health and wellness information. If designated by the defined rules, the service provider may initiate alerts, notifications, or calls to various individuals, entities, or devices. Such notifications, alerts, or calls, may include designated parts of the monitored health and wellness information as well as relevant information associated with a user of the portable device.

The foregoing description of exemplary implementations provides illustration and description, but is not intended to be exhaustive or to limit the embodiments described herein to the precise form disclosed. Modifications and variations are possible in light of the above teachings or may be acquired from practice of the embodiments.

For example, various features have been mainly described above with respect to a mobile or portable device configured to performing health and wellness monitoring functions. In other implementations, features described herein may be implemented mainly in one or more devices remote from the mobile or portable device, such as in-home medical or security devices.

Further, while series of blocks have been described with respect to FIGS. 7-9, the order of the acts may be varied in other implementations. Moreover, non-dependent acts may be implemented in parallel.

It will also be apparent that various features described above may be implemented in many different forms of software, firmware, and hardware in the implementations illustrated in the figures. The actual software code or specialized control hardware used to implement the various features is not limiting. Thus, the operation and behavior of the features of the invention were described without reference to the specific software code—it being understood that one would be able to design software and control hardware to implement the various features based on the description herein.

Further, certain features described above may be implemented as “logic” that performs one or more functions. This logic may include hardware, such as one or more processors, microprocessors, application specific integrated circuits, or field programmable gate arrays, software, or a combination of hardware and software.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

No element, act, or instruction used in the description of the present application should be construed as critical or essential to the invention unless explicitly described as such. Also, as used herein, the article “a” is intended to include one or more items. Where only one item is intended, the term “one” or similar language is used. Further, the phrase “based on” is intended to mean “based, at least in part, on” unless explicitly stated otherwise.

What is claimed is:

1. A method, comprising:
receiving wellness information from a mobile device;
comparing the received wellness information against a monitoring profile associated with the mobile device, the monitoring profile including a number of rules and each rule including defined criteria and a result event to be executed when the defined criteria are met;
determining whether the received wellness information matches the defined criteria in one of the number of rules; and
executing the result event when the received wellness information matches the defined criteria in one of the number of rules.

2. The method of claim 1, wherein the number of rules comprises at least two rules, with each rule including defined criteria based on different values of a same category of information, wherein a result event associated with the first rule is different that a result event associated with the second rule.

3. The method of claim 2, wherein the wellness information includes at least timestamp information and geographic location information.

4. The method of claim 3, wherein the same category of information comprises one of time of day, day of week, or geographic location information.

5. The method of claim 1, wherein receiving wellness information from a mobile device further comprises:
periodically transmitting a request for the wellness information to the mobile device; and
receiving the wellness information in response to the request.

6. The method of claim 1, wherein receiving wellness information from a mobile device further comprises:
periodically receiving the wellness information from a mobile device.

7. The method of claim 1, wherein receiving wellness information from a mobile device, further comprises:
receiving the wellness information via a wireless radio network.

8. The method of claim 1, wherein the result event for each of the number of rules comprises at least one of a notification or call initiation to an entity.

9. The method of claim 8, wherein the notification comprises at least one of a text message transmitted via a radio network, an email or instant message transmitted via a data network, or an alert transmitted via a video distribution network.

10. The method of claim 8, further comprising:
transmitting an on duty message to a device associated with the entity indicating an on duty status of the entity based on the monitoring profile,
wherein receipt of the on duty message causes the device associated with the entity to display an on duty indicator.

11. The method of claim 10, wherein the on duty indicator comprises a graphical indicator or an audible indicator.

12. The method of claim 8, wherein the call initiation comprises at least one of initiating a call between the mobile device and a call center interface, initiating a call between the mobile device and a caregiver, initiating a call between the mobile device and medical personnel, initiating an automated call to the caregiver, or initiating an automated call to the medical personnel.

13. The method of claim 1, further comprising:
receiving the monitoring profile;
receiving a request to modify the monitoring profile;
determining whether the request to modify the monitoring profile is received from an authorized user; and
providing a monitoring profile modification interface when the request to modify the monitoring profile is received from an authorized user.

14. The method of claim 13, wherein the request to modify the monitoring profile is received from the mobile device via a radio network or a data network.

15. The method of claim 1, further comprising:
identifying monitoring profile suggestions; and
providing the identified monitoring profile suggestions to a user associated with the monitoring profile.

16. The method of claim 15, wherein identifying monitoring profile suggestions, further comprises:
identifying a pattern of activity associated with the mobile device;
determining whether the identified pattern of activity matches any of a number of monitoring profile suggestions; and
identifying a selected monitoring profile suggestion when the identified pattern of activity matches any of a number of monitoring profile suggestions.

17. A system, comprising:
a portable wellness monitoring device; and
a service provider configured to:
receive wellness information from the portable wellness monitoring device via a radio network;
receive a monitoring profile associated with the portable wellness monitoring device, the monitoring profile including a number of rules, each rule including defined criteria and a result event to be executed when the defined criteria are met;
compare the received wellness information against the monitoring profile;
determine whether the received wellness information matches the defined criteria in one of the number of rules; and
execute the result event when the received wellness information matches the defined criteria in one of the number of rules.

18. The system of claim 17, wherein the portable wellness monitoring device comprises:
a wearable device configured to communicate with the service provider via the radio network.

19. The system of claim 17, wherein each of the number of rules in the monitoring profile comprises:
a days field indicating one or more week days to which the rule is to apply;
an hours field indicating hours during which the rule is to be applied;
a condition field indicating a wellness monitoring conditions;
a location field indicating a geographic location corresponding to the rule; and
a result field indicating the result event to be performed when the received wellness information matches the days field, the hours field, the condition field, and the location field.

20. The system of claim 19, wherein a first rule indicates a first result event and a second rule indicates a second result event, where the first rule and the second rule differ by one of the condition field and the location field value, and the result field value.

21. The system of claim 20, where the differing one field value comprises the location field, such that identical conditions in different locations result in different result events.

22. The system of claim 17, further comprising:
a notification receiving device to receive notifications associated with the result events,
wherein the service provider is further configured to:
transmit an on duty message to the notification receiving device indicating an on duty status of the notification receiving device,
wherein receipt of the on duty message causes the notification receiving device to display an on duty indicator.

23. The system of claim 17, wherein the portable wellness monitoring device includes health condition monitoring logic configured to monitor and store health condition information corresponding to a user,
wherein the health condition information comprises at least one of: blood pressure, body temperature, blood sugar level, or heart rate.

24. The system of claim 17, further comprising
a call center interface configured to receive a call initiation request from the service provider,
wherein the call initiation request includes a request to call the portable wellness monitoring device, request to call medical service personnel, or request to call a caregiver associated with a user of the portable wellness monitoring device.

25. A method, comprising:
receiving a call center service request from a mobile device, wherein the call center service request includes monitoring data collected by the mobile device;
initiating a call between the user device and a call center based on the request;
forwarding the received monitoring data to the call center;
receiving, at the call center, a request for information from the mobile device; and
providing, in response to the request, information to the mobile device based on the received monitoring data.
